(19) United States
(12) Patent Application Publication
(10) Pub. No.: US 2016/0346409 A1
(43) Pub. Date: Dec. 1, 2016

(54) TARGETED MOLECULAR IMAGING CONTRAST AGENTS

(71) Applicant: MCMASTER UNIVERSITY, Hamilton (CA)

(72) Inventors: John VALLIANT, Hamilton (CA); Aimen ZLITNI, Hamilton (CA)

(21) Appl. No.: 15/117,944

(22) PCT Filed: Feb. 10, 2015
(86) PCT No.: PCT/CA2015/000077

§ 371 Date: Aug. 10, 2016

Related U.S. Application Data
(60) Provisional application No. 61/937,780, filed on Feb. 10, 2014.

(51) Int. Cl. A61K 49/22; A61K 47/48

(52) U.S. Cl. CPC A61K 49/223; A61K 49/221; A61K 47/48869; A61K 47/48369

(57) ABSTRACT

Novel ultrasound contrast agents are provided which are covalently linked to a bioorthogonal reactive group, and optionally further coupled to a corresponding bioorthogonal reactive group coupled with a targeting entity. Methods for targeted ultrasound imaging using such contrast agents are also provided comprising the steps of: 1) injecting the contrast agent into a patient and imaging the patient at a site of interest to detect the contrast agent, wherein the detection of the contrast agent indicates the presence of a target within the patient.

### FIELD OF THE INVENTION

The present invention pertains to the field of medical imaging. More particularly, the present invention relates to the development and use of contrast agents for ultrasound molecular imaging.

### BACKGROUND OF THE INVENTION

Ultrasound imaging remains one of the most extensively used medical imaging methods because of its high spatial and temporal sensitivity, low cost, portability and accessibility. Contrast-enhanced ultrasound using gas-filled microbubbles (MBs) has further enhanced the utility of ultrasound and created the opportunity to employ biomolecule-targeted derivatives for molecular imaging applications. Such ultrasound contrast agents are generally comprised of an inert gas such as a perfluorocarbon, surrounded by a lipid, synthetic polymer, or protein shell. The traditional approach to targeting MBs, which are typically 1-8 μm in diameter and therefore restricted to intravascular targets, has been to link biomolecules with high affinity for a specific protein to the outer shell through covalent bonds (e.g. amide) or strong non-covalent interactions such as biotin-streptavidin binding. These approaches, which have largely exploited antibody and peptide vectors, have demonstrated the ability to selectively localize MBs to sites of angiogenesis, inflammation and intravascular thrombus formation.

While pre-targeting methods for nanometer-sized materials such as nanoparticles and liposomes have been reported recently, this is not directly applicable to pre-targeting strategies for molecular tumour imaging using ultrasound.

Rather than using targeting vectors to localize conjugated prosthetic groups, new strategies for creating molecular imaging probes are being exploited that employ pre-targeting and bio-orthoganal coupling chemistry. In such cases, a targeting vector is administered first, allowing time for localization and clearance from non-target organs, followed by a fluorescent or radiolabeled coupling partner. The inverse-electron-demand Diels-Alder reaction between tetrazines and trans-cyclooctene (TCO) is an example of a highly selective and rapid bioorthogonal coupling reaction that has been used successfully to prepare a range of targeted nuclear and optical imaging probes. However, the methods for such a coupling reaction have not been shown to work with micron-sized materials like ultrasound contrast agents.

Therefore, there remains a need for a strategy to localize MB's to overcome current problems with targeting micron-sized MB's, whose large size and ability to bind only intravascular targets make it particularly challenging to achieve and maintain good contrast in a timeframe that aligns with the limited in vivo stability of MB's.

### SUMMARY OF THE INVENTION

A novel approach to ultrasound molecular imaging has now been developed that employs functionalized contrast agents that are highly selective.

Accordingly, in one aspect, an ultrasound imaging contrast agent is provided coupled to a bioorthogonal reactive group.

In another aspect, a method of ultrasound imaging for a target in a patient is provided comprising the steps of: 1) injecting a contrast agent that is covalently linked to a bioorthogonal complex coupled to a targeting entity into a patient and 2) imaging the patient at a site of interest to detect the contrast agent, wherein the detection of the contrast agent indicates the presence of the target within the patient.

In another aspect, a method for targeted medical imaging is provided. The method comprises the steps of: 1) contacting a biological sample with a targeting entity comprising a first bioorthogonal reactive group, wherein said targeting entity binds a target; 2) contacting the biological sample with a micron-sized contrast agent comprising a second bioorthogonal reactive group reactive with said first bioorthogonal reactive group, wherein said first and second bioorthogonal groups react to form a detectable complex, and 3) imaging the sample for bound detectable complex to detect the presence of the target cell in the sample.

These and other aspects will become apparent in the detailed description that follows by reference to the following figures.

### BRIEF DESCRIPTION OF THE FIGURES

FIG. 1 illustrates the synthetic route of biotin-tetrazine;

FIG. 2 is a schematic illustrating localization of tetrazine functionalized microbubbles (MB_{TZ}) and an intravascular target (VEGFR2) labeled with a trans-cyclooctene (TCO) modified antibody;

FIG. 3 graphically illustrates fluorescence intensity of VEGFR2(+) H520 cell lysates obtained following treatment of cells with (a) TCO-antiVEGFR2 followed by Biotin-tetrazine followed by FITC-antiBiotin, (b) biotin-antiVEGFR2 followed by FITC-antiBiotin, and (c) Biotin-tetrazine followed by FITC-antiBiotin;

FIG. 4 graphically illustrates an analysis of the number of MBs per cell based on relative area from the flow chamber adhesion assay following washing for (a) the MB_{Tz} to TCO-antiVEGFR2 tagged H520 cells (VEGFR2 +ve), (b) anti-VEGFR2 targeted MBs (MB_{V}) to H520 cells, (c) MB_{Tz} to untreated H520 cells, (d) MB_{Tz} to TCO-antiVEGFR2 treated A431 cells (VEGFR2 −ve), and (e) MB_{c} to TCO-antiVEGFR2 treated H520 cells;

FIG. 5 is a schematic of the parallel plate flow chamber assay used to test and visualize the binding of MBs to cancer cells under flow conditions that result in a shear rate of 100 sec⁻¹;

FIG. 6 graphically illustrates the results of a semi-quantitative analysis of the number of MBs bound per cell based on relative area from the flow chamber adhesion assay following washing for (a) MB_{Tz} binding to A431 cells pre-incubated with TCO-anti-uPAR, (b) MB_{Tz-TCO-anti-uPAR} binding to A431 cells, (c) MB_{Tz} binding to untreated A431 cells, (d) MB_{Tz} binding to TCO-anti-uPAR treated MCF7 (uPAR (−)) cells, and (e) MB_{C} binding to TCO-anti-uPAR-tagged A431 cells.

FIG. 7 graphically illustrates the results of a semi-quantitative analysis of the number of MBs bound per cell based on relative area from the flow chamber adhesion assay following washing for (a) MB_{Tz} binding to PSMA (+) PC3 cells treated with TCO-J591, (b) MB_{Tz-TCO-J591} binding to PSMA (+) PC3 cells, (c) MB_{Tz} binding to untreated PC3 cells, (d) MB_{Tz} binding to TCO-J591 treated PSMA (−) PC3 cells, (e) MB_{Tz-TCO-J591} binding to PSMA (−) PC3 cells and (f) MB_{C} binding to PSMA (+) PC3 cells treated with TCO-J591; and

FIG. 8 illustrates exemplary reactive bioorthogonal reactive groups.

### DETAILED DESCRIPTION OF THE INVENTION

A method for targeted ultrasound imaging comprising the steps of: 1) administering to a patient a targeting entity comprising a first bioorthogonal reactive group, wherein said targeting entity binds a target; 2) after a period of time sufficient for the targeting entity to localize to the target, administering to the patient a micron-sized contrast agent comprising a second bioorthogonal reactive group reactive with said first bioorthogonal reactive group, wherein said first and second bioorthogonal groups react to form a detectable complex, and 3) imaging the patient at a site of interest for the presence of the contrast agent, wherein detection of the contrast agent indicates the presence of the target in the patient.

The present method is useful for imaging a wide variety of targets, including cellular markers, e.g. cellular markers that can readily be accessed through the vascular system. The markers may be markers of a disease or pathological condition such as inflammation, cancer, heart abnormalities, atherosclerosis, angiogenesis, intravascular thrombus formation. Examples of particular markers include cell surface receptors indicative of angiogenesis, e.g. vascular endothelial growth factor receptor 2 (VEGFR2) and αᵥβ₃ integrin. Cell surface proteins and transmembrane proteins indicative of cancer include, for example, urokinase-type plasminogen activator receptor (uPAR) which is overexpressed on the surface of endothelial cancer cells, prostate specific membrane antigen (PSMA) which is over-expressed in prostate carcinoma as well as neovasculature in other solid tumors. Markers of inflammation include as cell adhesion molecules, like VCAM-1, ICAM-1, E-selectin and P-selectin.

The targeting entity (or targeting vector) is selected to specifically bind to the target, e.g. cell-surface or transmembrane proteins and/or receptors indicative of a target disease or pathological condition. Thus, the targeting entity may be, for example, an antibody (such as monoclonal or polyclonal antibodies), or other target-binding molecule such as receptor ligand. The targeting entity may be naturally-occurring or a synthetic entity which incorporates a specific binding modality for the target, e.g. a receptor binding site. Targeting entities may be readily obtained using established techniques in the art, e.g. generation of antibodies, or may be commercially available. Antibodies for targets of angiogenesis such as VEGFR2 include antibody EIC from Abcam (ab9530) and CD309 (BioLegend), and antibodies are also available for targets of inflammation and cancer. For targets of inflammation, PSLG-1 is a ligand for P-selectin, and targeting entities for αᵥβ₃ integrin include anti-human integrin αᵥβ₃ monoclonal antibody, e.g. MAB1976F, as well as RGD peptides. Glutamate-urea-lysine analogues, synthetic small molecules, are another example of a targeting entity for PSMA (prostate specific membrane antigen) in prostate carcinoma.

Suitable imaging contrast agents for use in the present method include ultrasound contrast agents. Generally such contrast agents are greater in size than nano-sized contrast agents, e.g. preferably, contrast agents which are about micro-sized, but which may be smaller by up to an order of magnitude (10⁻⁷ m). Examples of suitable contrast agents include ultrasound contrast agents such as microbubbles. Microbubbles for use as ultrasound contrast agents are generally 0.5-10 microns in size, and comprise a shell composed of protein, e.g. albumin, lysozyme; lipids; sugars, e.g. galactose or sucrose; surfactants such as SPAN-40 and TWEEN-40; polymers, e.g. styrene, poly-(D,L-lactide-co-glycolide) polymers (PGLA) such as PLGA-polyethelene glycol (PLGA-PEG) polymer, polyvinyl alcohol, polylactic acid polymers such as polyperfluorooctyloxycaronyl-poly(lactic acid) (PLA-PFO), multilayer (PEM) shells such as poly(allylamine hydrochloride) (PAH) and poly(styrene sulfonate) (PSS); or combinations thereof. Microbubbles are filled with a gas that provides them with the echogenicity required for their function as ultrasound contrast agents. Examples of microbubble gases include air, perfluorocarbon, octafluoropropane, sulphur hexafluoride, and nitrogen.

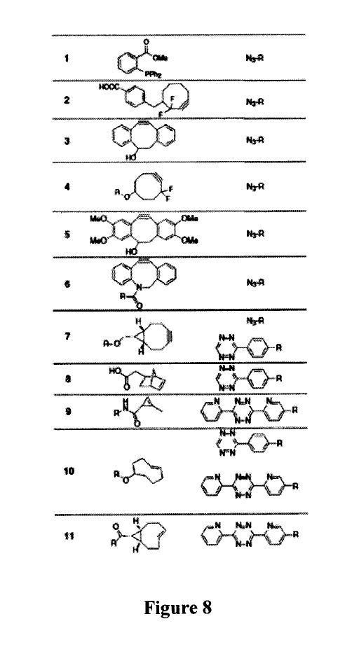

The targeting entity and contrast agent are each coupled or linked to a compound having a bioorthogonal reactive group, e.g. a compound having a first bioorthogonal reactive group and a compound having a second bioorthogonal reactive group, respectively. The bioorthogonal reactive groups react with one another to form a linkage, such as a covalent linkage, and thereby yield a bioorthogonal complex. The reaction of bioorthogonal reactive groups varies with each pair of bioorthogonal reactive groups. Examples of bioorthogonal reactive group pairs include tetrazine and transcyclooctene (TCO) reactive groups which react by an inverse-electron-demand Diels-Alder reaction, azide and with functionalized phosphine reactive groups (which react by a Staudinger ligation reaction), and azide and strained alkyne reactive groups (which react by a copper-free click reaction). Accordingly, examples of bioorthogonal reactive compounds include, but are not limited to, the tetrazine: 4-(1,2,4,5-tetrazin-3-yl)phenyl)methanamine hydrochloride, and the transcyclooctene: (E)-cyclooct-4-enyl-2,5-dioxopyrrolidin-1-yl carbonate (TCO-NHS); the azide: 2,5-dioxopyrrolidin-1-yl 2-azidoacetate (or NHS-azide) and the phosphine: 4-(2,5-dioxopyrrolidin-1-yl) 1-methyl 2-(diphenylphosphino)terephthalate (or NHS-Phosphine), and the NHS-azide and the strained alkyne: dimethoxyazacyclooctyne. Chemical structures of additional orthogonal reactive compound pairs are shown in FIG. 8. The first and second bioorthogonal reactive groups are interchangeable, for example, the first and second bioorthogonal reactive groups may be either a tetrazine or a transcyclooctene, except that they cannot both be a tetrazine or a transcyclooctene. When the first bioorthogonal reactive group is a tetrazine, the second bioorthogonal group is a transcyclooctene, and similarly, when the first bioorthogonal reactive group is a transcyclooctene, the second bioorthogonal group is a tetrazine. Thus, the targeting entity and contrast agent incorporate corresponding bioorthogonal groups, i.e. bioorthogonal groups that react with one another to form a complex.

As one of skill in the art will appreciate, the targeting entity and contrast agent may be coupled or linked to corresponding bioorthogonal reactive groups using various techniques. For example, coupling agents may be used to link a compound having a bioorthogonal reactive group, including biotin-streptavidin coupling agents, carbodiimide or maleimide coupling, or vinyl sulfone coupling agents, to the targeting entity or the contrast agent in a manner generally familiar to the skilled person. In some cases, the shell of the contrast agent permits covalent direct coupling of the bioorthogonal reactive group by chemical activation without the use of additional coupling agents, e.g. polymer shells (e.g. PLGA-polyethelene glycol polymer shell) are actived to include reactive chemical groups such as amides to permit coupling with a bioorthogonal reactive group.

In a first step of the method, a biological sample is contacted with the targeting entity which is linked to a first bioorthogonal reactive group. For use in vivo, the targeting entity is administered by intravascular injection such that the targeting entity will be able to bind to any existing target within a patient. The targeting entity must be formulated into an administrable form, e.g. admixed with a physiologically acceptable carrier. The term “physiologically acceptable” refers to its acceptability for use in the pharmaceutical and veterinary arts, i.e. not being unacceptably toxic or otherwise unsuitable for physiological use. Examples of suitable carriers include aqueous solutions in sterile and pyrogen-free form, optionally buffered or made isotonic. The carrier may be distilled water, a carbohydrate-containing solution (e.g. dextrose) or a saline solution comprising sodium chloride and optionally buffered. An amount of targeting entity is administered that would yield sufficient quantity of bioorthogonal complex for imaging purposes. In one embodiment, an amount in the range of 0.1-100 mg/kg of a targeting entity such as an antibody may be administered.

Following administration of the targeting entity and a sufficient period of time for the targeting entity to localize to the intended target site, e.g. site of inflammation or angiogenesis, or to a tumour site, such as a period of at least 12-24 hours, the contrast agent comprising a second bioorthogonal reactive group is administered to the patient in a manner similar to that used for targeting entity. The contrast agent is similarly formulated for intravascular administration in a physiological acceptable carrier. Following injection of the contrast agent in an amount sufficient to react with the targeting entity, and a sufficient period of time for the contrast agent to localize and for the bioorthogonal reactive groups to react, e.g. a period of about 2-30 minutes, preferably 4-10 minutes, the patient may be imaged, e.g. using ultrasound, in a region of interest to detect the presence of any bioorthogonal complex formed by detection of the contrast agent. Detection of complex indicates that the target is present, and that the target disease or condition is present. In one embodiment, the amount of contrast agent administered is in the range of about 0.1×10⁹ microbubbles/g to 1×10¹⁰ microbubbles/kg.

In another embodiment, the targeting entity linked to a first bioorthogonal reactive group may be first coupled to the second bioorthogonal reactive group linked to the contrast agent. This complex may then be formulated for administration to a patient and administered to the patient, as described, for imaging. Following a sufficient period of time to permit localization of the complex, imaging of the area of interest within the patient may be conducted as above.

The present method may also be used to delivery therapeutic agents to target sites. For example, the contrast agent, e.g. microbubble, may be modified to incorporate a therapeutic agent. In this regard, therapeutic agents such a nucleic acid, proteins and other agents, may be conjugated to or within the shell of the microbubble. Preferred therapeutic agents include those which treat diseases or pathological conditions which are beneficially treated by access to the vascular system, and thus, which are effectively delivered by in accordance with the present methods using targeting entities such as those exemplified herein, such as inflammation, cancer, heart abnormalities, atherosclerosis, angiogenesis, and intravascular thrombus formation. Following administration of therapeutic-loaded microbubbles, localization through reaction of the bioorthogonal reactive groups, the application of ultrasound sufficient to burst the microbubble, e.g. sonoporation, will release the therapeutic.

In a further aspect, a kit is provided for use in targeted ultrasound imaging. The kit may comprise a contrast agent, e.g. microbubble, coupled to a bioorthogonal reactive group, either directly or via a coupling agent. In this case, the kit may also provide a bioorthogonal reactive group that corresponds with that coupled to the contrast agent that may then be coupled to any desired targeting entity, or a corresponding bioorthogonal reactive group that is already coupled to a targeting entity. Alternatively, the kit may include a contrast agent coupled to a bioorthogonal complex, e.g. a first bioorthogonal reactive group covalently linked to a second bioorthogonal reactive group. The bioorthogonal complex may optionally be linked to a specific targeting entity, e.g. an antibody or ligand, for a specific target of a particular disease or condition. Alternatively, the bioorthogonal complex is not linked to a specific targeting entity and, thus, may be bound to any desired targeting entity. The kit will additionally include instructions for conducting the present method.

Embodiments of the invention are described by reference to the following specific examples which are not to be construed as limiting.

### EXAMPLE 1

### Use of Tetrazine Microbubbles to Target VEGFR2-Expressing Cells

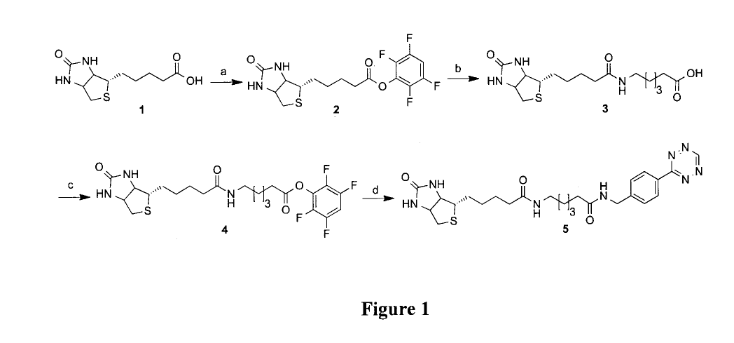

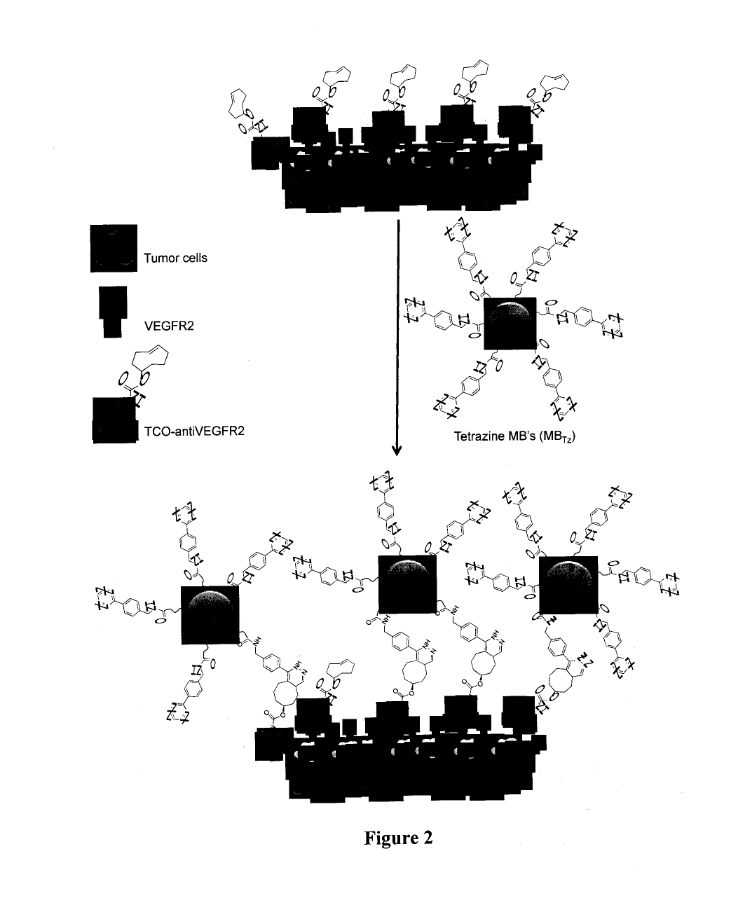

To demonstrate the feasibility of capturing micron-sized bubbles, a novel tetrazine-tagged microbubble (MB_{Tz}) was developed (FIG. 1) and its reactivity towards cells treated with a transcyclooctene (TCO)-conjugated anti-vascular endothelial growth factor receptor 2 (VEGFR2) antibody was evaluated (FIG. 2). VEGFR2 is overexpressed on tumor cells and upon activation triggers multiple signalling pathways that contribute to angiogenesis. The choice of this target also allows for the use of anti-VEGFR2-tagged MB's (MB_{V}) developed by Willmann et al. (Radiology 2008, 246, 508-518) as a convenient tool to validate the tetrazine-TCO capture methodology against a conventional targeting approach.

Tetrazine-functionalized bubbles were prepared using commercially available streptavidin coated MB's (MicroMarker Target-Ready contrast agents, VisualSonics) and a biotinylated tetrazine. The biotin-tetrazine derivative was synthesized from biotin in four high yielding steps as shown in FIG. 1 using the reagents and conditions as follows for each step: a) 2,3,5,6-tetrafluorophenyl trifluoroacetate, DMF, TEA, 30 min, 95%; b) 6-amino-hexanoic acid, DMF, TEA, 75° C., 12 h, 91%; c) 2,3,5,6-tetrafluorophenyl trifluoroacetate, DMF, DMSO, 80° C., 1 h, 96%; d) 4-(1,2,4,5-tetrazin-3-yl)phenyl)methanamine hydrochloride, DMF, TEA, 1 h, 75%. DMF=dimethylformamide, TEA=triethylamine, DMSO=dimethylsulfoxide. The desired product was ultimately obtained by coupling commercially available 4-(1,2,4,5-tetrazin-3-yl)phenyl)methanamine hydrochloride (6.2 mg, 0.033 mmol; Sigma-Aldrich) with 6-biotinamidohexanoic TFP ester (25 mg, 0.049 mmol) at room temperature. After semi-preparative HPLC, the biotin-tetrazine derivative was isolated in a 75% yield. The product was stable in the freezer for more than 6 months. The TCO-conjugated antibody (TCO-antiVEGFR2) was prepared by combining an excess (20 equiv.) of commercially available (E)-cyclooct-4-enyl-2,5-dioxopyrrolidin-1-yl carbonate (TCO-NHS) with antiVEGFR2 (eBioscience) at 4° C. overnight at pH 9-9.5. After purification using a 30 kDa centrifugal filter (Amicon Ultra-0.5) MALDI-TOF MS showed the product had an average of 3 TCO derivatives per antibody.

The derivatized bubbles (MB_{Tz} and MB_{V}) were prepared by adding the biotin-tetrazine derivative or biotinylated-antiVEGFR2 to freshly reconstituted streptavidin-coated MBs. Isolation of the bubbles from the biotin-containing reagents was accomplished by treating the solution with streptavidin-coated magnetic beads (New England Biolabs), which bound residual tetrazine and antibody, followed by simple magnetic separation. This approach has been found to be more convenient than centrifugation and washing as it minimizes the amount of direct handling of the MBs.

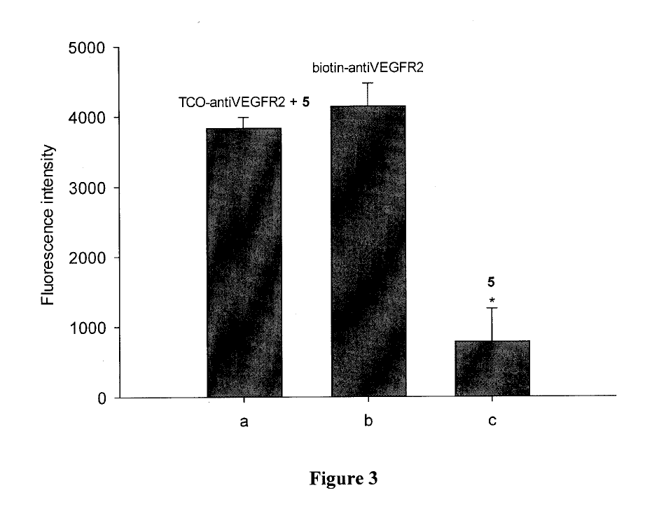

Prior to working with MB_{Tz}, the ability of the biotin-tetrazine derivative to bind to VEGFR2-positive H520 cells tagged with TCO-anti-VEGFR2 was evaluated in vitro in direct comparison to a commercially available biotinylated anti-VEGFR2 antibody (biotin-anti-VEGFR2). The biotin-tetrazine derivative was added to H520 cells that had been incubated with TCO-antiVEGFR2 and the extent of tetrazine-TCO conjugation determined by adding a FITC labelled anti-biotin antibody (FITC-anti-Biotin) and measuring the arising fluorescence from cell lysates. As a control, FITC-anti-Biotin was added to H520 cells that had been incubated with a comparable amount of biotin-antiVEGFR2. The tetrazine-TCO construct (FIG. 3a) showed effectively identical intensity to direct tagging with the biotinylated antibody (FIG. 3b). The binding of the biotin-tetrazine derivative and FITC-anti-Biotin to H520 cells in the absence of any VEGFR2 antibodies was measured and showed significantly lower intensity (FIG. 3c) indicating minimal non-specific binding.

To evaluate the effectiveness of the tetrazine-TCO capture strategy, MBs were evaluated initially in vitro under flow conditions (as opposed to simply in culture) similar to that found in tumor capillaries using a parallel plate flow chamber system (Glycotech, Rockville, Md.). VEGFR2-expressing cells (H520) and cells lacking VEGFR2 (A431) were incubated with TCO-anti-VEGFR2 30 min prior to the assay. Using a syringe pump, cells were washed with PBS for 2 min to remove any unbound antibody followed by either functionalized or unmodified MBs for 4 min at a 100 sec⁻¹ shear rate. To eliminate any non-specifically bound MBs, cells were subsequently washed with PBS for 2 min at a 10-fold higher (1000 sec⁻¹) shear rate. Optical microscopy was used to visualize the plates where videos were taken during the flow assay and static images for analysis acquired after the final washing step was completed.

Qualitatively, the tetrazine modified MBs could be seen concentrating to a significant extent on H520 cells (VEGFR2(+)) that had been pre-incubated with TCO-anti-VEGFR2. A relatively small amount of MBs could be seen bound non-specifically to the flow chamber during the dynamic component of all assays, which were removed after the final washing step. Microscopy-images (Brightfield) taken subsequently exhibited significant retention of MB_{Tz} on TCO-anti-VEGFR2 tagged H520 cells compared to experiments run in untreated cells. Repeating the study using VEGFR2 negative A431 cells treated with TCO-anti-VEGFR2 showed little retention of functionalized MBs.

To compare with traditional targeting strategies, binding of anti-VEGFR2-tagged MBs (Willmann et al. 2008) on VEGFR2-expressing H520 cells was evaluated under identical conditions and showed comparable binding that exhibited by MB_{Tz} on TCO-anti-VEGFR2 tagged H520 cells. To confirm that TCO-anti-VEGFR2 did not promote non-specific binding of the MBs to the cells, unmodified MBs as a control (MB_{C}) were exposed to TCO-anti-VEGFR2 tagged H520 cells and negligible MB retention was observed.

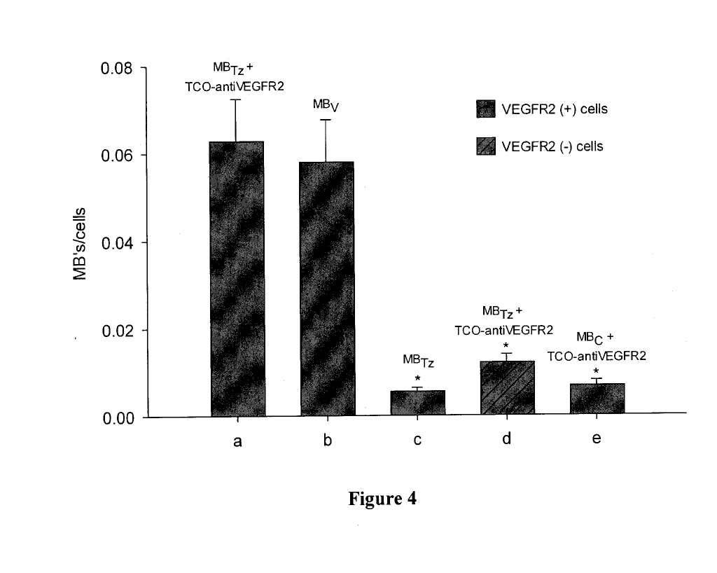

A semi-quantitative analysis was performed by comparing the area covered by the MBs (black spheres) in each image to the area covered by the cells determined using an open source image processing package (Schindelin et al. Nat. Methods 2012, 9, 676-682). Prior to the analysis, solution concentrations and sizes of the MBs were determined using a Coulter Counter to ensure comparable test conditions. The MB_{C}, MB_{Tz} and MB_{V} concentrations were similar at 5.7×10⁶, 6.9×10⁶ and 9.4×10⁶ MBs/mL, respectively, as were the average sizes, at 2.62±0.73, 3.11±0.85 and 2.68±0.73 μm, respectively. MB_{Tz} binding to TCO-antiVEGFR2 tagged H520 cells (FIG. 4a) was over an order of magnitude higher than MB_{Tz} binding to unlabelled cells (FIG. 4c). Minimal binding of MB_{C} to TCO-antiVEGFR2 tagged H520 cells (FIG. 4e) and MB_{Tz} to VEGFR2 negative TCO-antiVEGFR2 tagged A431 cells (FIG. 4d) was observed which is consistent with the microscopy images. The tetrazine system exhibited similar binding to the previously reported anti-VEGFR2-tagged MBs (MB_{V}) (FIG. 4b) indicating the pre-targeting strategy has at least the equivalent capacity to localize contrast agent to the VEGFR2 target.

Having demonstrated successful capture in vitro under flow conditions similar to that found in tumour capillaries, a preliminary study in animal models was undertaken. Ultrasound images were performed in mammals using CD1 nu/nu mice bearing SKOV-3 (VEGFR2(+)) human adenocarcinoma tumours. TCO-antiVEGFR2 in PBS was administered via the tail vein 100 μg/200 μL. Twenty four hours later, to allow adequate time for accumulation of the targeting entity in the tumour, MB_{Tz} was administered (approximately 6×10⁷ MBs/70 μL saline). Four minutes post injection, transverse color-coded parametric non-linear contrast mode ultrasound images obtained using a destruction replenishment sequence (as described in Willmann et al. 2008) and differential signal enhancement with VevoCQ quantification software (VisualSonics). Regions of interest were based on the vascularity of the tumours determined from the initial distribution of the MBs following injection.

The images showed high retention of MB_{Tz} in vascularized regions of the SKOV-3 tumors. Even in cases where the tumours were poorly vascularized, providing less surface area for capture, contrast enhancement was significant. The contrast obtained by TCO-antiVEGFR2/MB_{TZ} treatment was greater than that of images obtained in animals that were not administered the antiVEGFR2 antibody and in A431 (VEGFR2(−)) tumour models to which antiVEGFR2 was administered. Localization of biotinylated anti-VEGFR2 modified MBs was also apparent.

The results presented represent the first evidence that capturing MBs in vitro and in vivo using bioorthogonal coupling chemistry is feasible. Taken together, the flow chamber assays and imaging data demonstrate that localization of MBs is related to the presence of the target and the tetrazine-TCO reaction and not simply formation of antibody-labelled bubbles in situ. The comparable binding observed for the bubble capture strategy and the known VEGFR2 targeted MBs (MBᵥ) further validates that the reported approach can be used to selectively visualize a specific target in a flow format or in animal models with simple microscopy and ultrasound imaging, respectively.

### EXAMPLE 2

### Use of Tetrazine Microbubbles to Target uPAR-Expressing Cells

The ability to target tetrazine-functionalized MBs (MB_{Tz}) to uPAR-expressing cells using the strategy described in Example 1 was also tested.

TCO-modified antibody was prepared as generally described in (Zlitni et al. Angew. Chem. Int. Ed. Engl. 2014, 53, 6459-6463). Briefly, the pH of anti-uPAR antibody (American Diagnostica Inc., 3936) (450 μL, 225 μg, 1.5 nmol) was adjusted to 9 by adding 3 μL of 1M Na₂CO₃(aq) before adding (E)-cyclooct-4-enyl-2,5-dioxopyrrolidin-1-yl carbonate (TCO-NHS, 8 μg, 30 nmol, 20 eq) in DMSO (4 μL). The solution was left on a shaker overnight at 4° C. The desired product was isolated from excess TCO using an Amicon Ultra-0.5 Centrifugal filter (30 kDa) and washed with PBS three times.

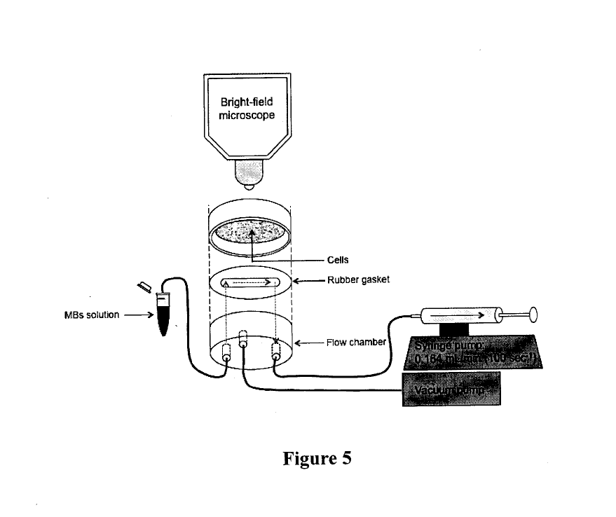

MB_{Tz} and TCO-conjugated antibody (TCO-anti-uPAR) were prepared as reported previously (Zlitni et al. 2014). The antibody used was a monoclonal antibody against human uPAR (CD 87) and conjugated to TCO following the procedure described in Example 1. The binding of MB_{Tz} to uPAR-expressing cancer cells (A431) was studied in two different strategies in a flow chamber adhesion assay (FIG. 5). In the first approach, the cells were incubated with TCO-anti-uPAR for 30 min prior to administering MB_{Tz}. While the second approach, MB_{Tz} was incubated with TCO-anti-uPAR (MB_{Tz-anti-uPAR}) for 20 min before administering to cells. As a control, the binding of MB_{Tz} to cancer cells lacking uPAR (MCF7 cells) was assessed as well as the binding of non-labeled MBs (MB_{C}) to pre-treated A431 cells.

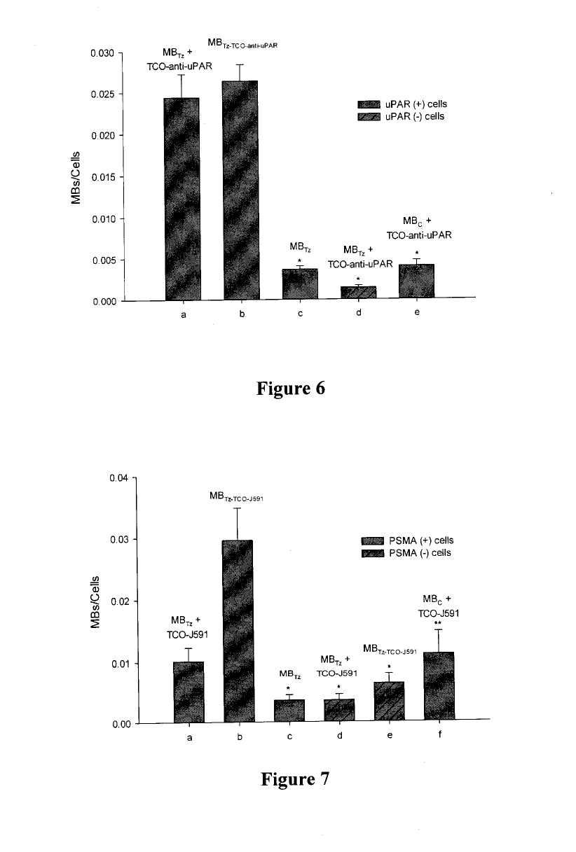

In the flow chamber adhesion assay, cells were washed with 1 mL PBS before administering any type of MB. To further validate the efficacy of the binding and to wash any non-specifically bound MBs, cells were washed with 2 mL PBS at a 10-fold increased flow rate. After the washing step, static images were obtained using Bright-field microscopy at different fields of view and further analyzed using FIJI software. Qualitatively, the greatest MB binding was exhibited in the targeting strategies, e.g. when A431 cells were incubated with TCO-anti-uPAR followed by MB_{Tz}, or when MB_{Tz} was incubated with TCO-anti-uPAR (MB_{Tz-anti-uPAR}) and then administered to A431 cells (uPAR (+)). Minimal binding of MB_{Tz} was seen on untreated A431 cells (uPAR (+)), as well as pre-treated TCO-anti-uPAR MCF7 cells (uPAR (−)). Unmodified MBs (MB_{C}) were also evaluated on pre-treated TCO-anti-uPAR A431 cells (uPAR (+)) and showed negligible binding. A semi-quantitative analysis was performed using an open source image analysis software (FIJI) where the area covered by the MBs was measured and divided over the area covered by the cells in each image. Similarly, the binding of MBs in targeting strategies (of FIGS. 6a and 6b) showed at least 6-fold higher binding than the negative controls (of FIGS. 6c, d, and e).

### EXAMPLE 3

### Use of Tetrazine Microbubbles to Target PSMA-Expressing Cells

Prostate specific membrane antigen (PSMA), which is a transmembrane glycoprotein, is highly expressed in prostate carcinoma as well as neovasculature in other solid tumors. The ability to target MB_{Tz} to PSMA-expressing cells was examined. The antibody used for targeting was J591 anti-PSMA antibody. J591 is a monoclonal antibody that binds the extracellular domain of PSMA and was kindly provided by the laboratory of Dr. Neil Bander (Department of Urology, New York Presbyterian Hospital-Weill Medical College of Cornell University). The TCO-modified antibody was prepared as described in Example 2. Briefly, the pH of J591 antibody (500 μL, 250 μg, 1.67 nmol) was adjusted to 9 by adding 3 μL of 1M Na₂CO₃ (aq) before adding (E)-cyclooct-4-enyl-2,5-dioxopyrrolidin-1-yl carbonate (TCO-NHS, 17.8 μg, 66.8 nmol, 40 eq) in DMSO (9 μL). The solution was left on a shaker overnight at 4° C. The desired product was isolated from excess TCO using an Amicon Ultra-0.5 Centrifugal filter (30 kDa) and washed with PBS three times.

Following the same flow chamber adhesion assay procedure mentioned above, the adhesion of MB_{Tz} to transfected PSMA-expressing PC3 cells and to PSMA-lacking PC3 cells was assessed. Preliminary results from the flow chamber assay and Bright-field microscopy (20×) showed binding of MB_{Tz} to PSMA (+) PC3 cells when MB_{Tz} was pre-incubated with TCO-J591 for 20 min (MB_{Tz-TCO-J591}) before the assay (FIG. 7b), while less binding was shown when the cells were incubated with TCO-J591 for 30 min before introducing MB_{Tz} (FIG. 7a). This is probably due to the fast internalization of TCO-J591 in the cells making the TCO moiety unreachable by MB_{Tz}. In control experiments, minimal binding of MB_{Tz} to untreated PSMA(+) PC3 cells (FIG. 7c) as well as to treated PSMA(−) PC3 cells (FIG. 7d,e) was observed. Negligible binding of MB_{C} was observed on treated PSMA(+) PC3 cells (FIG. 7f).

### Materials, Instruments and General Information for Examples

Microbubbles (MBs) were obtained using MicroMarker™ Target-Ready Contrast Agent Kit (VisualSonics Inc., Toronto, Canada; 8.4×10⁸ MBs/vial). Streptavidin coated magnetic beads (New England BioLabs) and MACSiMAG™ Separator (MiltenyiBiotec) magnet were used during the purification of MBs. Conjugated-antibodies were analyzed on a MALDI Bruker Ultraflextreme Spectrometer. MB size and concentration were determined using Z2 Coulter counter (Beckman Coulter, Fullerton, Calif.).

Preparation of Microbubbles (MBs). Streptavidin coated MBs (MicroMarker Target-Ready contrast agents, VisualSonics) were reconstituted in 500 μL sterile saline (0.9% sodium chloride) according to the manufacturer's instructions. To prepare the tetrazine-coated MBs (MB_{Tz}), biotin-Tz (FIG. 1) (70 μg, 1.35×10⁴ mmol) in 50 μL of saline:MeOH (1:1 v/v) was added dropwise to the reconstituted MBs. After 45 min, 200 μL of the bottom of the solution was removed carefully with minimal agitation of the bubbles and was discarded. Streptavidin coated magnetic beads (200 μL) were added and after 20 min, 200 μL of solution was removed carefully and discarded and the sample placed beside a magnet. After decanting the solution, MBs were rinsed with 200 μL saline and then transferred to another vial. MB_{Tz-TCO-antibody} was prepared by incubating 50 μL of MB_{Tz} solution with 20 μL of TCO-antibody (10 μg) for 20 min before running the experiment.

Cells and Culture Methods. A431 (CRL-1740) cells were cultured in DMEM media supplemented with 10% fetal bovine serum and 1% penicillin streptomycin. MCF7 (HTB-22) cells were cultured in Eagle's Minimum Essential Medium (EMEM) supplemented with 10% fetal bovine serum and 1% penicillin streptomycin. Transfected PC3 cells that express and don't express PSMA were cultured in F 12-K media supplemented with 10% fetal bovine serum, 1% penicillin streptomycin and 0.1% geneticin. The cell lines were maintained at 37° C. under 5% CO₂.

Flow Chamber Cell Adhesion Assay. The flow assay was as generally described by Zlitni et al. 2014. Cells (8×10⁵) were plated separately in 30 mm Corning tissue culture dishes 2 days prior to running the assay. For MB_{Tz} and associated controls, cells were incubated with TCO-antibody (30 μg/mL) for 30 min prior to running the assay. The parallel-plate flow chamber (Glycotech, Rockville, Md.) was setup as shown in FIG. 2. Using a syringe pump (PhD 2000, Harvard Apparatus, Holliston, USA) cells were first rinsed with 1 mL PBS, 1 mL of MBs solution at a wall shear rate of 100 sec⁻¹ (flow rate=0.164 mL/min) and subsequently with 2 mL PBS at 1000 sec⁻¹ shear rate. Binding of MBs was visualized using a Celestron PentaView LCD Digital Brightfield S4 Microscope with 20× objective. Images were recorded and the extent of binding assessed by comparing the area covered by MBs to the total area covered by cells in each image using image analysis (FIJI) software.

Relevant portions of references referred to herein are incorporated by reference.

What is claimed:

1. A micron-sized contrast agent coupled to a bioorthogonal reactive group.

2. The contrast agent of claim 1, wherein the bioorthogonal reactive group is covalently bound to a corresponding bioorthogonal reactive group to form a bioorthogonal complex.

3. The contrast agent of claim 2, wherein the bioorthogonal complex is linked to a targeting entity.

4. The contrast agent of claim 1, which is a microbubble having a shell comprising protein, lipid, sugar, polymers, polyelectrolytes or combinations thereof.

5. The contrast agent of claim 2, wherein the bioorthogonal reactive group and the corresponding bioorthogonal reactive group, in either order, are a tetrazine and a transcyclooctene, or an azide and a functionalized phosphine, or an azide and a strained alkyne.

6. The contrast agent of claim 3, wherein the targeting entity is an antibody or a receptor ligand.

7. A method for targeted ultrasound imaging comprising the steps of: 1) injecting a contrast agent as defined in claim 3 into a patient and 2) imaging the patient using ultrasound to detect the contrast agent, wherein the detection of the contrast agent indicates the presence of the target within the patient.

8. The method of claim 7, wherein the bioorthogonal reactive group and the corresponding bioorthogonal reactive group, in either order, are a tetrazine and a transcyclooctene, or an azide and a functionalized phosphine, or an azide and a strained alkyne.

9. The method of claim 7, wherein the target is a cellular marker for one of inflammation, cancer, a heart abnormality, atherosclerosis, angiogenesis, and intravascular thrombus formation.

10. The method of claim 9, wherein the marker is selected from the group consisting of vascular endothelial growth factor receptor 2 (VEGFR2), αᵥβ₃ integrin, urokinase-type plasminogen activator receptor (uPAR), prostate specific membrane antigen (PSMA), VCAM-1, ICAM-1, E-selectin and P-selectin.

11. The method of claim 7, wherein the targeting entity is an antibody or a receptor ligand.

12. A method for targeted ultrasound imaging comprising the steps of: 1) administering to a patient a targeting entity comprising a first bioorthogonal reactive group, wherein said targeting entity binds a target; 2) after a period of time sufficient for the targeting entity to localize to the target, administering to the patient a micron-sized contrast agent comprising a second bioorthogonal reactive group reactive with said first bioorthogonal reactive group, wherein said first and second bioorthogonal groups react to form a detectable complex, and 3) imaging the patient at a site of interest for the presence of the contrast agent, wherein detection of the contrast agent indicates the presence of the target in the patient.

13. The method of claim 12, wherein the bioorthogonal reactive group and the corresponding bioorthogonal reactive group, in either order, are a tetrazine and a transcyclooctene, or an azide and a functionalized phosphine, or an azide and a strained alkyne.

14. The method of claim 12, wherein the target is a cellular marker for one of inflammation, cancer, a heart abnormality, atherosclerosis, angiogenesis, and intravascular thrombus formation.

15. The method of claim 14, wherein the marker is selected from the group consisting of vascular endothelial growth factor receptor 2 (VEGFR2), αᵥβ₃ integrin, urokinase-type plasminogen activator receptor (uPAR), prostate specific membrane antigen (PSMA), VCAM-1, ICAM-1, E-selectin and P-selectin.

16. The method of claim 15, wherein the targeting entity is an antibody or a receptor ligand.

17. The contrast agent of claim 5, wherein the tetrazine is 4-(1,2,4,5-tetrazin-3-yl)phenyl)methanamine hydrochloride, and the transcyclooctene is (E)-cyclooct-4-enyl-2,5-dioxopyrrolidin-1-yl carbonate.

18. The contrast agent of claim 1, additionally comprising a therapeutic agent.

19. A method of delivering a therapeutic agent to a target site in a patient comprising administering to the patient a contrast agent as defined in claim 3, wherein the contrast agent further comprises the therapeutic agent.
